(19) United States
(12) Patent Application Publication
(10) Pub. No.: US 2020/0060551 A1
(43) Pub. Date: Feb. 27, 2020

(54) SYSTEM, IMAGE PROCESSING APPARATUS, MEASUREMENT CONTROL METHOD, AND IMAGE PROCESSING METHOD

(71) Applicants: CANON KABUSHIKI KAISHA, Tokyo (JP); KEIO UNIVERSITY, Tokyo (JP)

(72) Inventors: Hiroki Kajita, Tokyo (JP); Nobuaki Imanishi, Tokyo (JP); Sadakazu Aiso, Tokyo (JP); Moemi Urano, Tokyo (JP); Kenichi Nagae, Yokohama-shi (JP); Kazuhito Oka, Tokyo (JP)

(21) Appl. No.: 16/542,681

(22) Filed: Aug. 16, 2019

(30) Foreign Application Priority Data
Aug. 24, 2018 (JP) 2018-157800

(51) Int. Cl. A61B 5/00; A61B 8/08

(52) U.S. Cl. CPC A61B 5/0095; A61B 5/0071; A61B 8/481; A61B 5/0033; A61B 8/5207; A61B 5/0075

(57) ABSTRACT

A system includes an image acquiring unit configured to acquire a first image generated by imaging fluorescence that is generated by emitting excitation light onto a subject into which a fluorescent contrast agent has been introduced; and a photoacoustic measuring unit configured to implement photoacoustic measurement by receiving a photoacoustic wave generated in response to light emission onto the subject, wherein the photoacoustic measuring unit is further configured to control the photoacoustic measurement on the basis of the first image.

### BACKGROUND OF THE INVENTION

### Field of the Invention

The present disclosure relates to a system for performing photoacoustic imaging.

### Description of the Related Art

Photoacoustic imaging (also known as “optical ultrasound imaging”) using a contrast agent may be used for examining a blood vessel, a lymph vessel or the like. WO 2017/022337 describes a photoacoustic image generation apparatus in which a contrast agent used to enhance the contrast of a lymph node, a lymph vessel, or the like is set as an evaluation subject, and light is emitted onto the contrast agent at a wavelength at which the contrast agent absorbs the light so that a photoacoustic wave is generated.

Another known technique for examining a lymph vessel or the like is a fluorescence imaging method, in which a contrast agent introduced into a lymph vessel or the like is irradiated with excitation light and an image of fluorescence produced by the contrast agent is formed.

With the photoacoustic imaging described in WO 2017/022337 or a fluorescence imaging method, however, it may be difficult to ascertain the structure of a contrast enhancement target (for example, the course of a blood vessel, a lymph vessel, or the like) in the interior of a subject.

### SUMMARY OF THE INVENTION

An object of the present disclosure is to provide a system for generating an image from which it is easy to ascertain the structure of a contrast enhancement target using photoacoustic imaging.

The first aspect of the present disclosure is a system, including: an image acquiring unit configured to acquire a first image generated by imaging fluorescence that is generated by emitting excitation light onto a subject into which a fluorescent contrast agent has been introduced; and a photoacoustic measuring unit configured to implement photoacoustic measurement by receiving a photoacoustic wave generated in response to light emission onto the subject, wherein the photoacoustic measuring unit is further configured to control the photoacoustic measurement on the basis of the first image.

The second aspect of the present disclosure is an image processing apparatus, including: a first image acquiring unit configured to acquire a first image generated by imaging fluorescence that is generated by emitting excitation light onto a subject into which a fluorescent contrast agent has been introduced; a second image acquiring unit configured to acquire a second image generated on the basis of a photoacoustic wave that is generated in response to light emission onto the subject; and a synthesizing unit configured to generate a synthesized image by synthesizing the first image with the second image.

The third aspect of the present disclosure is a measurement control method, including: acquiring a first image generated by imaging fluorescence that is generated by emitting excitation light onto a subject into which a fluorescent contrast agent has been introduced; and controlling, on the basis of the first image, photoacoustic measurement in which a photoacoustic wave generated in response to light emission onto the subject is received.

The fourth aspect of the present disclosure is an image processing method including: acquiring a first image generated by imaging fluorescence that is generated by emitting excitation light onto a subject into which a fluorescent contrast agent has been introduced; acquiring a second image generated on the basis of a photoacoustic wave that is generated in response to light emission onto the subject; and generating a synthesized image by synthesizing the first image with the second image.

According to the present disclosure, it is possible to provide a system for generating an image from which it is easy to ascertain the structure of a contrast enhancement target using photoacoustic imaging.

Further features of the present invention will become apparent from the following description of exemplary embodiments with reference to the attached drawings.

### BRIEF DESCRIPTION OF THE DRAWINGS

FIG. 1 is a block diagram of a system according to an embodiment;

FIG. 2 is a block diagram showing a specific example of an image processing apparatus according to this embodiment and configurations on the periphery thereof;

FIG. 3 is a detailed block diagram of a photoacoustic apparatus according to this embodiment;

FIG. 4 is a schematic diagram of a probe according to this embodiment;

FIG. 5 is a flowchart of an image processing method according to this embodiment;

FIGS. 6A to 6D are illustrative views of a method for determining a photoacoustic imaging range according to this embodiment;

FIGS. 7A to 7D are contour maps of a calculated value of formula (1) corresponding to a contrast agent when a combination of wavelengths is varied;

FIG. 8 is a line graph showing the calculated value of formula (1) corresponding to the contrast agent when a concentration of ICG is varied;

FIG. 9 is a graph showing respective molar absorption coefficient spectra of oxyhemoglobin and deoxyhemoglobin;

FIG. 10 is a view showing a GUI according to this embodiment;

FIGS. 11A and 11B are spectroscopic images of a right forearm extensor when the concentration of ICG is varied;

FIGS. 12A and 12B are spectroscopic images of a left forearm extensor when the concentration of ICG is varied; and

FIGS. 13A and 13B are spectroscopic images of the inside of a lower right thigh and the inside of a lower left thigh when the concentration of ICG is varied.

### DESCRIPTION OF THE EMBODIMENTS

A preferred embodiment of the present disclosure will be described below with reference to the figures. Note, however, that dimensions, materials, shapes, relative arrangements, and so on of constituent components described below may be modified as appropriate in accordance with the configuration of the apparatus to which the disclosure is applied and various conditions. Accordingly, the scope of this disclosure is not limited to the following description.

A photoacoustic image acquired using a subject information acquisition system according to the present disclosure reflects an absorption amount and an absorption rate of optical energy. The photoacoustic image expresses a spatial distribution of at least one type of subject information, such as an acoustic pressure (an initial acoustic pressure) at which a photoacoustic wave is generated, a light absorption energy density, and a light absorption coefficient. The photoacoustic image may be an image expressing a two-dimensional spatial distribution or an image (volume data) expressing a three-dimensional spatial distribution. The system according to this embodiment generates a photoacoustic image by imaging a subject into which a contrast agent has been introduced. Note that in order to ascertain the three-dimensional structure of the contrast enhancement target, the photoacoustic image may be either an image expressing a two-dimensional spatial distribution in a depth direction from the surface of the subject, or may express a three-dimensional spatial distribution.

Further, the system according to the present disclosure is capable of generating a spectroscopic image of the subject using a plurality of photoacoustic images corresponding to a plurality of wavelengths. The spectroscopic image of the present disclosure is generated using photoacoustic signals corresponding respectively to a plurality of wavelengths and based on photoacoustic waves generated by irradiating the subject with light of a plurality of different wavelengths.

The spectroscopic image may indicate the concentration of a specific substance in the subject, which concentration may be generated using photoacoustic signals corresponding respectively to a plurality of wavelengths. When a light absorption coefficient spectrum of the used contrast agent differs from the light absorption coefficient spectrum of the specific substance, an image value of the contrast agent on the spectroscopic image differs from an image value of the specific substance on the image. Hence, a region of the contrast agent can be distinguished from a region of the specific substance in accordance with the image values of the spectroscopic image. The specific substance may be a substance constituting the subject, such as hemoglobin, glucose, collagen, melanin, fat, or water. Likewise in this case, a contrast agent having a different light absorption spectrum to the light absorption coefficient spectrum of the specific substance is selected. Moreover, depending on the type of the specific substance, the spectroscopic image may be calculated using a different calculation method.

In the following embodiment, a spectroscopic image having an image value that is calculated using oxygen saturation calculation formula (1) below will be described. The inventors discovered that a calculated value Is(r) deviates greatly from a numerical range of the oxygen saturation of hemoglobin, if a measurement value I(r) of a photoacoustic signal acquired using a contrast agent in which the wavelength dependency of the light absorption coefficient exhibits a different tendency to that of oxyhemoglobin and deoxyhemoglobin is substituted into formula (1) for calculating the oxygen saturation of hemoglobin in the blood (or an index having a correlation to the oxygen saturation) on the basis of photoacoustic signals corresponding respectively to a plurality of wavelengths. Therefore, by generating a spectroscopic image having the calculated value Is(r) as an image value, a region of hemoglobin (a blood vessel region) inside the subject can easily be separated or distinguished on the image from a region in which the contrast agent exists (a lymph vessel region, for example, when the contrast agent is introduced into a lymph vessel).

$$Is ( r ) = \frac{\frac{{I}^{{λ}_{2}} ( r )}{{I}^{{λ}_{1}} ( r )} \cdot {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{I}^{{λ}_{2}} ( r )}{{I}^{{λ}_{1}} ( r )} \cdot ( {ɛ}_{HbO}^{{λ}_{1}} - {ɛ}_{Hb}^{{λ}_{1}} )} \qquad ( 1 )$$

Here, I₁^{λ}(r) is a calculated value based on a photoacoustic wave generated by emitting light of a first wavelength λ₁, and I₂^{λ}(r) is a calculated value based on a photoacoustic wave generated by emitting light of a second wavelength λ₂. ε_{Hb1}^{λ} is the molar absorption coefficient [mm⁻¹ mol⁻¹] of deoxyhemoglobin corresponding to the first wavelength λ₁, and ε_{Hb2}^{λ} is the molar absorption coefficient [mm⁻¹ mol⁻¹] of deoxyhemoglobin corresponding to the second wavelength λ₂. ε_{HbO1}^{λ} is the molar absorption coefficient [mm⁻¹ mol⁻¹] of oxyhemoglobin corresponding to the first wavelength λ₁, and ε_{HbO2}^{λ} is the molar absorption coefficient [mm⁻¹ mol⁻¹] of oxyhemoglobin corresponding to the second wavelength λ₂. r denotes a position. The calculated values I₁^{λ}(r), I₂^{λ}(r) may also be absorption coefficients μₐ₁^{λ}(r), μₐ₂^{λ}(r) or initial acoustic pressures P₀₁^{λ}(r), P₀₂^{λ}(r).

When a calculated value based on the photoacoustic wave generated from the hemoglobin region (the blood vessel region) is substituted into formula (1), the oxygen saturation of the hemoglobin (or an index having a correlation to the oxygen saturation) is acquired as the calculated value Is(r). Meanwhile, in the case of a subject into which a contrast agent has been introduced, when a calculated value based on an acoustic wave generated from the contrast agent region (the lymph vessel region, for example) is substituted into formula (1), a virtual concentration distribution of the contrast agent is acquired as the calculated value Is(r). Note that, when calculating the concentration distribution of the contrast agent using formula (1), the same numerical value of the molar absorption coefficient of the hemoglobin can be used. On a spectroscopic image having the image value Is(r) acquired in this manner, the hemoglobin region (the blood vessel) and the contrast agent region (a lymph vessel, for example) inside the subject can both be depicted in a separable (distinguishable) state.

In this embodiment, the images value of the spectroscopic image is calculated using formula (1) for calculating the oxygen saturation, but when an index other than the oxygen saturation is calculated as the image value of the spectroscopic image, a calculation method other than formula (1) may be used. Any known indices and calculation methods can be used as the index and the calculation method, and therefore detailed description thereof has been omitted.

Further, in the system according to the present disclosure, the spectroscopic image may be an image expressing a ratio of a first photoacoustic image based on a photoacoustic wave generated by emitting light of the first wavelength λ₁ to a second photoacoustic image based on a photoacoustic wave generated by emitting light of the second wavelength λ₂. In other words, the spectroscopic image may be an image based on the ratio of the first photoacoustic image, which is based on a photoacoustic wave generated by emitting light of the first wavelength λ₁, to the second photoacoustic image, which is based on a photoacoustic wave generated by emitting light of the second wavelength λ₂. Any image generated in accordance with a modification of formula (1) can also be expressed by the ratio of the first photoacoustic image to the second photoacoustic image and may therefore be regarded as an image (a spectroscopic image) based on the ratio of the first photoacoustic image to the second photoacoustic image.

Further, in order to ascertain the three-dimensional structure of the contrast enhancement target, the spectroscopic image may be an image expressing a two-dimensional spatial distribution in the depth direction from the surface of the subject, or may express a three-dimensional spatial distribution.

A configuration and an image processing method of the system according to this embodiment will be described below.

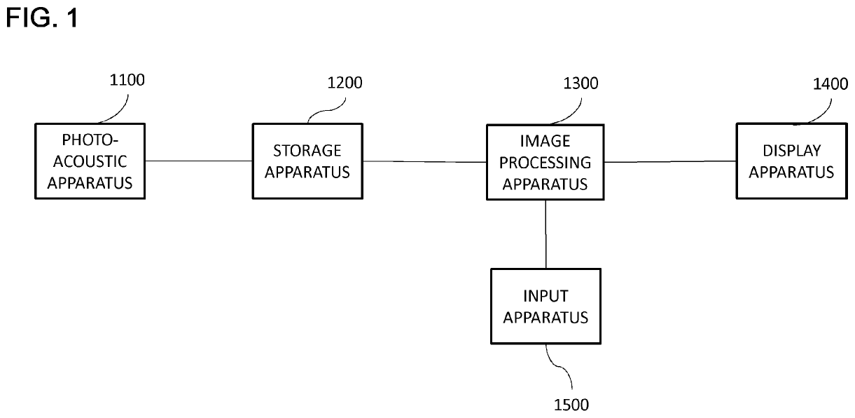

The system according to this embodiment will be described using FIG. 1. FIG. 1 is a block diagram showing the configuration of the system according to this embodiment. The system according to this embodiment includes a photoacoustic apparatus 1100, a storage apparatus 1200, an image processing apparatus 1300, a display apparatus 1400, and an input apparatus 1500. Data may be transmitted and received between the apparatuses either by wire or wirelessly.

The photoacoustic apparatus 1100 generates a photoacoustic image by imaging a subject into which a contrast agent has been introduced, and outputs the generated photoacoustic image to the storage apparatus 1200. The photoacoustic apparatus 1100 generates information indicating characteristic values corresponding respectively to a plurality of positions in the subject using reception signals acquired by receiving photoacoustic waves generated as a result of light emission. In other words, the photoacoustic apparatus 1100 generates a spatial distribution of characteristic value information derived from photoacoustic waves as medical image data (photoacoustic images). The photoacoustic apparatus 1100 according to this embodiment is configured to also be capable of fluorescence imaging, and an imaging range of the photoacoustic image is determined on the basis of a fluorescent image.

The storage apparatus 1200 may be a storage medium such as a ROM (Read Only Memory), a magnetic disk, or a flash memory. The storage apparatus 1200 may also be a storage server connected via a network such as a PACS (Picture Archiving and Communication System).

The image processing apparatus 1300 processes the photoacoustic images stored in the storage apparatus 1200 and information such as supplementary information attached to the photoacoustic images.

Units assuming a calculation function of the image processing apparatus 1300 may be constituted by a processor such as a CPU or a GPU (Graphics Processing Unit) and a calculation circuit such as an FPGA (Field Programmable Gate Array) chip. These units may be constituted by a single processor and a single calculation circuit or by a plurality of processors and a plurality of calculation circuits.

A unit assuming a storage function of the image processing apparatus 1300 may be constituted by a non-transitory storage medium such as a ROM (Read Only Memory), a magnetic disk, or a flash memory. The unit assuming the storage function may also be a volatile medium such as a RAM (Random Access Memory). Non-transitory storage medium may be used as a storage medium storing a program. Further, the unit assuming the storage function may be constituted by either a single storage medium or a plurality of storage media.

A unit assuming a control function of the image processing apparatus 1300 is constituted by an arithmetic element such as a CPU. The unit assuming the control function controls operations of the respective components of the system. The unit assuming the control function may control the respective components of the system upon reception of instruction signals generated in response to various operations, such as a measurement start operation, from an input unit. The unit assuming the control function may also control the operations of the respective components of the system by reading program code stored in a computer 150.

The display apparatus 1400 is a liquid crystal display, an organic EL (Electro Luminescence) display, or the like. Further, the display apparatus 1400 may display a GUI for manipulating an image or operating an apparatus.

The input apparatus 1500 is an operation console constituted by a mouse, a keyboard, or the like that can be operated by a user, for example. Alternatively, the display apparatus 1400 may be constituted by a touch panel so that the display apparatus 1400 can be used as the input apparatus 1500.

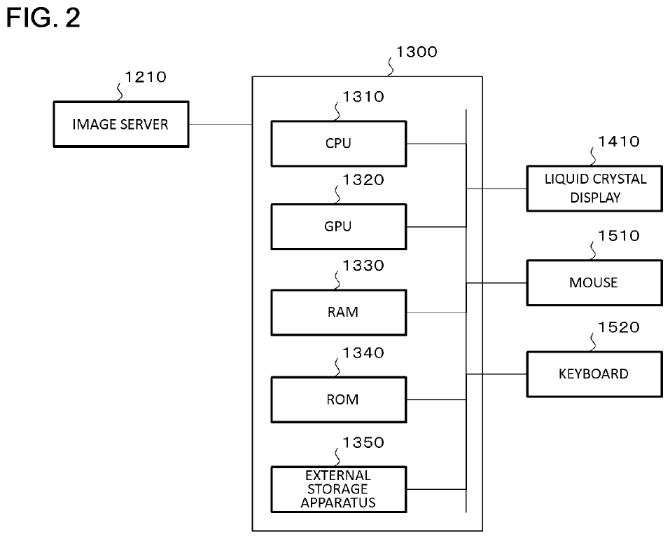

FIG. 2 shows an example of a specific configuration of the image processing apparatus 1300 according to this embodiment. The image processing apparatus 1300 according to this embodiment is constituted by a CPU 1310, a GPU 1320, a RAM 1330, a ROM 1340, and an external storage device 1350. Further, a liquid crystal display 1410 functioning as the display apparatus 1400 and a mouse 1510 and a keyboard 1520 functioning as the input apparatus 1500 are connected to the image processing apparatus 1300. Furthermore, the image processing apparatus 1300 is connected to an image server 1210 such as a PACS (Picture Archiving and Communication System) functioning as the storage apparatus 1200. Thus, image data can be stored on the image server 1210, and the image data on the image server 1210 can be displayed on the liquid crystal display 1410.

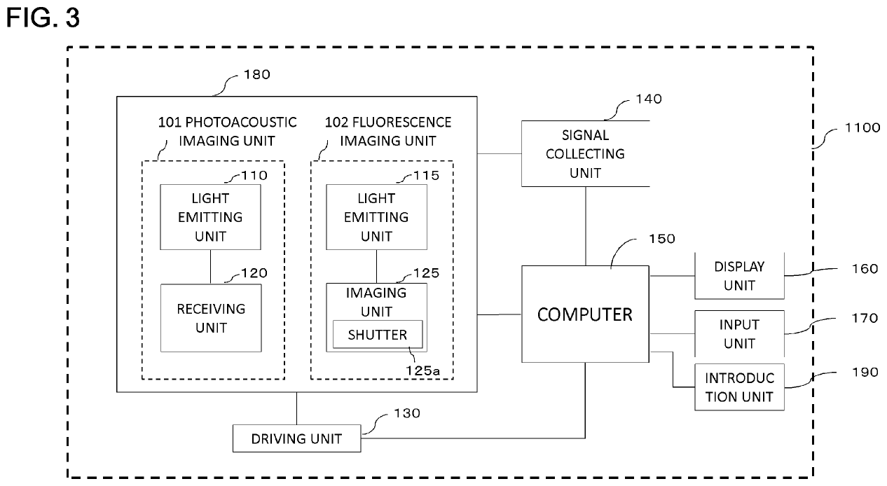

Next, an example configuration of one of the apparatuses included in the system according to this embodiment will be described. FIG. 3 is a schematic block diagram of one of the apparatus included in the system according to this embodiment.

The photoacoustic apparatus 1100 according to this embodiment includes a driving unit 130, a signal collecting unit 140, the computer 150, a probe 180, and an introduction unit 190. The probe 180 includes a photoacoustic imaging unit 101 having a light emitting unit 110 and a receiving unit 120, and a fluorescence imaging unit 102 having a light emitting unit 115 and an imaging unit 125. In this embodiment, the photoacoustic imaging unit 101, the driving unit 130, the signal collecting unit 140, and the computer 150 together constitute a photoacoustic measuring unit for executing photoacoustic measurement, in which a photoacoustic wave generated by irradiating a subject with light is received. Photoacoustic measurement includes a series of measurement processes from irradiating the subject with light to receiving the photoacoustic wave. Further, when the photoacoustic measuring unit includes the driving unit 130, as in this embodiment, the photoacoustic measurement also includes moving the receiving unit in order to receive the photoacoustic wave.

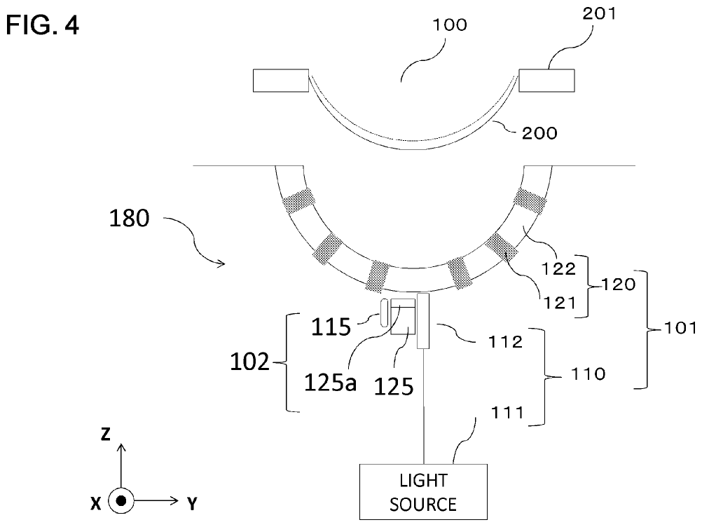

FIG. 4 is a schematic diagram showing the probe 180 according to this embodiment. The measurement target is a subject 100 into which a contrast agent has been introduced by the introduction unit 190. The driving unit 130 executes a mechanical scan by driving the light emitting unit 110, the receiving unit 120, the light emitting unit 115, and the imaging unit 125. The light emitting unit 110 emits light onto the subject 100, whereby an acoustic wave is generated inside the subject 100. An acoustic wave generated by a photoacoustic effect caused by light is also known as a photoacoustic wave. The receiving unit 120 receives the photoacoustic wave and outputs an electric signal (a photoacoustic signal) in the form of an analog signal. The light emitting unit 115 emits excitation light for exciting the fluorescent contrast agent onto the subject. The contrast agent, which is excited by the excitation light, emits fluorescence. The imaging unit 125 captures a fluorescent image of the contrast agent and outputs an electric signal (a fluorescent image signal) in the form of an analog signal.

The signal collecting unit 140 converts the analog signals output from the receiving unit 120 and the imaging unit 125 into digital signals and outputs the digital signals to the computer 150. The computer 150 stores the digital signals output from the signal collecting unit 140 as signal data derived from the photoacoustic wave and signal data relating to the fluorescent image.

The computer 150 specifies the position of a lymph vessel from the fluorescent image and determines a position for capturing a photoacoustic image along the course of the lymph vessel. The computer 150 generates a photoacoustic image by executing signal processing on the stored digital signals. Further, the computer 150 implements image processing on the acquired photoacoustic image and then outputs the photoacoustic image to the display unit 160. The display unit 160 displays an image based on the photoacoustic image. The display image is stored in a memory inside the computer 150 or the storage apparatus 1200, which is provided in a data management system or the like connected by a modality and a network, on the basis of a storage instruction from the user or the computer 150.

The computer 150 also performs drive control on configurations included in the photoacoustic apparatus. The computer 150 controls photoacoustic measurement by the photoacoustic apparatus. Further, the display unit 160 may display a GUI or the like as well as images generated by the computer 150. The input unit 170 is configured so that the user can input information therein. Using the input unit 170, the user can perform operations such as starting and terminating measurement and issuing an instruction to store a created image.

The respective components of the photoacoustic apparatus 1100 according to this embodiment will now be described in detail.

<Light Emitting Unit 110>

The light emitting unit 110 includes a light source 111 for emitting light and an optical system 112 for guiding the light emitted from the light source 111 to the subject 100. The light emitted from the light source 111 includes pulsed light with a so-called rectangular wave, triangular wave, or the like.

Considering the thermal confinement condition and the stress confinement condition, a pulse width of the light emitted by the light source 111 may be equal to or below 100 ns. Further, the wavelength of the light may be within a range of approximately 400 nm to 1600 nm. To capture a high-resolution image of a blood vessel, a wavelength at which absorption by the blood vessel is high (for example, a wavelength of between 400 nm and 700 nm, inclusive) may be used. To capture an image of a deep part of an organism, light of a wavelength at which absorption by background tissue (water, fat, and so on) of the organism is generally low (for example, a wavelength of between 700 nm and 1100 nm, inclusive) may be used.

The light source 111 is a laser, a light-emitting diode, or the like. Further, when measurement is performed using light of a plurality of wavelengths, a tunable or wavelength-variable light source may be used. In a case of irradiating the subject with a plurality of wavelengths, a plurality of light sources for generating light of different wavelengths may be prepared so that the subject can be irradiated from the respective light sources alternately. In this disclosure, even in a case where a plurality of light sources are used, the light sources will be referred to collectively as the light source in the singular form. Various lasers, such as a solid-state laser, a gas laser, a dye laser, or a semiconductor laser can be used as the laser. For example, a pulse laser such as an Nd:YAG laser or an alexandrite laser may be used as the light source. Alternatively, a Ti:sa laser or an OPO (Optical Parametric Oscillator) laser that uses Nd:YAG laser light as excitation light may be used as the light source. Alternatively, a flash lamp or a light-emitting diode may be used as the light source 111. Furthermore, a microwave source may be used as the light source 111.

Optical elements such as lenses, mirrors, and optical fiber can be used in the optical system 112. When the subject 100 is a breast or the like, a light emitting unit of the optical system may be constituted by a diffusion plate or the like for diffusing the pulsed light so that the light is emitted with a widened beam diameter. In a photoacoustic microscope, meanwhile, in order to increase the resolution, the light emitting unit of the optical system 112 may be constituted by a lens or the like, and the beam may be emitted after being focused.

In other embodiments, the optical system 112 may be omitted from the light emitting unit 110 and light may be emitted onto the subject 100 directly from the light source 111.

<Receiving Unit 120>

The receiving unit 120 includes a transducer 121 that outputs an electric signal upon reception of an acoustic wave, and a support 122 for supporting the transducer 121. Further, the transducer 121 may be constituted by a transmitting unit for transmitting the acoustic wave. The transducer functioning as the receiving unit and the transducer functioning as the transmitting unit may be constituted by a single (common) transducer or a plurality of separate transducers.

The transducer 121 may include a piezoelectric ceramic material such as PZT (lead zirconate titanate), a piezoelectric polymer such as PVDF (polyvinylidene fluoride), or the like. An element other than a piezoelectric element may also be used for the transducer. A transducer using a capacitive transducer (a CMUT: Capacitive Micro-machined Ultrasonic Transducer) or the like, for example, can be used. Any transducer may be used as long as the transducer is capable of outputting an electric signal upon reception of an acoustic wave. Moreover, the signal acquired from the transducer is a time-resolved signal. In other words, the amplitude of the signal acquired from the transducer expresses a value based on the acoustic pressure (for example, a value that is commensurate with the acoustic pressure) received by the transducer at each time.

A frequency component of the photoacoustic wave is generally between 100 KHz and 100 MHz, and therefore a transducer capable of detecting these frequencies may be used as the transducer 121.

The support 122 may be constituted by a metallic material having high mechanical strength or the like. To ensure that a large amount of the emitted light enters the subject, mirror surface processing or light-scattering processing may be performed on the surface of the support 122 facing the subject 100. In this embodiment, the support 122 has the shape of a hemispherical shell and is configured so that a plurality of transducers 121 can be supported on the hemispherical shell. In this case, orientation axes of the transducers 121 disposed on the support 122 gather near the curvature center of the hemisphere. Thus, when an image is formed using signals output from the plurality of transducers 121, the image quality improves near the curvature center. Note that as long as the support 122 is capable of supporting the transducers 121, any configuration may be used. The plurality of transducers may be arranged on the support 122 in a plane or a curve so as to form a so-called 1D array, 1.5D array, 1.75D array, or 2D array. The plurality of transducers 121 correspond to a plurality of receiving units.

Furthermore, the support 122 may function as a container storing an acoustic matching material. In other words, the support 122 may be used as a container for disposing an acoustic matching material between the transducers 121 and the subject 100.

Further, the receiving unit 120 may include an amplifier for amplifying a time series of analog signals output from the transducer 121. The receiving unit 120 may also include an A/D converter for converting the time series of analog signals output from the transducer 121 into a time series of digital signals. In other words, the receiving unit 120 may include the signal collecting unit 140 to be described below.

A space between the receiving unit 120 and the subject 100 is filled with a medium through which photoacoustic waves can propagate. The material of the medium may be selected as the material with highest transmittance for the photoacoustic wave among materials through which acoustic waves can propagate and whose acoustic characteristic match to those of the subject 100 and the transducers 121 on the interface. Examples of the medium are water, ultrasound gel, and the like.

<Light Emitting Unit 115>

The light emitting unit 115 emits excitation light for exciting the contrast agent. A light-emitting diode or a laser diode may be used as a light source of the light emitting unit 115. The excitation light supplied from the light emitting unit 115 may have a wavelength capable of exciting a fluorescent pigment of the contrast agent. When the contrast agent is ICG, the wavelength of the excitation light is within a range of 760-800 nm, for example. The light emitting unit 115 may also include a white LED for emitting white light in order to capture visible images as well as fluorescent images.

<Imaging Unit 125>

The imaging unit 125 captures a fluorescent image emitted from the subject. The imaging unit 125 captures the fluorescent image using a color CCD camera capable of acquiring two-dimensional images, for example. The wavelength of the fluorescence of ICG is 800-850 nm, and therefore, when ICG is used as the contrast agent, an infrared observation camera having a reception sensitivity within this wavelength range is used. The imaging unit 125 also includes a notch filer for cutting (removing) reflection light from the excitation light. When the contrast agent is ICG, the wavelength of the excitation light is 760-800 nm, and therefore the notch filter removes wavelengths within this range while transmitting other wavelengths. By transmitting visible light at or below 760 nm, the imaging unit 125 can also capture a visible image under white light.

The imaging unit 125 further includes a mechanical shutter 125a for protecting the imaging unit 125 from the light pulse emitted from the light emitting unit 110 for the purpose of photoacoustic imaging. The light pulse emitted from the light emitting unit 110 is powerful, and therefore the imaging unit 125 may break if the light pulse enters the imaging unit 125 as is. Hence, the mechanical shutter 125a is controlled in synchronization with an emission timing (a photoacoustic wave acquisition timing) of the light emitting unit 110 so as to be closed at least while the light emitting unit 110 emits the light pulse. Instead of using the mechanical shutter, an infrared cut filter may be used. Further, a light amount suppression unit does not have to block entrance of the light pulse emitted from the light emitting unit 110 completely and may be configured as desired as long as the amount of light entering the imaging unit 125 can be suppressed to an extent at which the imaging unit 125 does not break. The mechanical shutter 125a and the infrared cut filter are examples of a light amount suppression unit for suppressing the amount of light entering the imaging unit 125.

The imaging unit 125 may implement imaging within a non-emission period between light pulses emitted intermittently by the light emitting unit 110. For example, the light emitting unit 110 may emit light pulses between approximately 10 and 20 times per second, and the imaging unit 125 may implement imaging during a non-emission period between the emitted light pulses. Alternatively, the imaging unit 125 may implement imaging in a state where light emission by the light emitting unit 110 is temporarily stopped.

FIG. 4 is a side view of the probe 180. The probe 180 according to this embodiment includes the receiving unit 120 in which the plurality of transducers 121 are arranged three-dimensionally on the hemispherical support 122, which includes an opening. Further, a light emitting unit of the optical system 112 is disposed on a bottom portion of the support 122. The light emitting unit 115 and the imaging unit 125 are also disposed on the bottom portion of the support 122. Note that the light emitting unit 115 and the imaging unit 125 are positioned so as not to block the light emitted from the light emitting unit of the optical system 112. In this embodiment, the photoacoustic imaging unit 101 (the light emitting unit 110 and the receiving unit 120) and the fluorescence imaging unit 102 (the light emitting unit 115 and the imaging unit 125) are provided integrally on the single probe 180, but these components may be disposed separately and driven (moved) individually.

In this embodiment, as shown in FIG. 4, the subject 100 contacts a holding unit 200 so that the shape thereof is maintained.

A space between the receiving unit 120 and the holding unit 200 is filled with a medium through which photoacoustic waves can propagate. The material of the medium may be selected as the material with highest transmittance for the photoacoustic wave among materials through which acoustic waves can propagate and whose acoustic characteristic match to those of the subject 100 and the transducers 121 on the interface. Examples of the material are water, ultrasound gel, and the like.

The holding unit 200 functioning as a holding unit maintains the shape of the subject 100 during measurement. By holding the subject 100 in the holding unit 200, movement of the subject 100 can be suppressed and the position of the subject 100 can be held within the holding unit 200. A resin material such as polycarbonate, polyethylene, or polyethylene terephthalate can be used as the material of the holding unit 200.

The holding unit 200 is attached to an attachment unit 201. The attachment unit 201 may be configured so that a plurality of types of holding units 200 can be exchanged in accordance with the size of the subject. For example, the attachment unit 201 may be configured so that holding units having different curvature radii, curvature centers, and so on can be exchanged.

<Driving Unit 130>

The driving unit 130 changes the relative positions of the subject 100, the receiving unit 120, and so on. The driving unit 130 may include a motor such as a stepping motor for generating driving force, a driving mechanism for transmitting the driving force, and a position sensor for detecting position information relating to the receiving unit 120. The driving mechanism may be a leadscrew mechanism, a link mechanism, a gear mechanism, a hydraulic mechanism, or the like. Further, the position sensor may be a position meter or the like using an encoder, a variable resistor, a linear scale, a magnetic sensor, an infrared sensor, an ultrasound sensor, and so on.

The driving unit 130 is not limited to changing the relative positions of the subject 100 and the receiving unit 120 in an XY direction (in two dimensions) and may change the relative positions in one dimension or three dimensions.

Further, as long as the driving unit 130 can change the relative positions of the subject 100 and the receiving unit 120, the receiving unit 120 may be fixed and the subject 100 may be moved. When the subject 100 is moved, a configuration whereby the subject 100 is moved by moving the holding unit holding the subject 100 or the like may be adopted. Alternatively, the subject 100 and the receiving unit 120 may both be moved.

The driving unit 130 may move the relative positions either continuously or in a step-and-repeat fashion. The driving unit 130 may be an electric stage that moves the subject 100 and/or the receiving unit 120 along a programmed locus or a manual stage.

Further, in this embodiment, the driving unit 130 performs a scan by driving the light emitting unit 110 and the receiving unit 120 simultaneously, but either the light emitting unit 110 or the receiving unit 120 may be driven alone.

The probe 180 may be a hand-held type probe provided with a grip portion, and in this case the photoacoustic apparatus 1100 need not include the driving unit 130.

<Signal Collecting Unit 140>

The signal collecting unit 140 includes an amplifier for amplifying the electric signals serving as the analog signals output from the transducers 121, and an A/D converter for converting the analog signals output from the amplifier into digital signals. The digital signals output from the signal collecting unit 140 are stored in the computer 150. The signal collecting unit 140 may also be referred to as a Data Acquisition System (DAS). In this disclosure, the term “electric signal” is a concept including both analog signals and digital signals. Note that a light detection sensor such as a photodiode may detect the light emitted from the light emitting unit 110, and the signal collecting unit 140 may start the processing described above in synchronization therewith, using the detection result as a trigger.

<Computer 150>

The computer 150, which functions as an information processing apparatus, is constituted by similar hardware to the image processing apparatus 1300. More specifically, units assuming a calculation function of the computer 150 may be constituted by a processor such as a CPU or a GPU (Graphics Processing Unit) and a calculation circuit such as an FPGA (Field Programmable Gate Array) chip. These units may be constituted by a single processor and a single calculation circuit or by a plurality of processors and a plurality of calculation circuits.

A unit assuming a storage function of the computer 150 may be constituted by a volatile medium such as a RAM (Random Access Memory). Non-transitory storage medium may be used as the storage medium storing a program. Further, the unit assuming the storage function of the computer 150 may be constituted by either a single storage medium or a plurality of storage media.

A unit assuming a control function of the computer 150 is constituted by an arithmetic element such as a CPU. The unit assuming the control function of the computer 150 controls operations of the respective components of the photoacoustic apparatus. The unit assuming the control function of the computer 150 may control the respective components of the photoacoustic apparatus upon reception of instruction signals generated in response to various operations, such as a measurement start operation, from the input unit 170. The unit assuming the control function of the computer 150 also controls the operations of the respective components of the photoacoustic apparatus by reading program code stored in the unit assuming the storage function. In other words, the computer 150 is capable of functioning as a control apparatus of the system according to this embodiment.

The computer 150 and the image processing apparatus 1300 may be constituted by the same hardware. A single piece of hardware may assume the functions of both the computer 150 and the image processing apparatus 1300. In other words, the computer 150 may assume the functions of the image processing apparatus 1300. Further, the image processing apparatus 1300 may assume the functions of the computer 150 functioning as an information processing apparatus.

<Display Unit 160>

The display unit 160 is a liquid crystal display, an organic EL (Electro Luminescence) display, or the like. Further, the display unit 160 may display a GUI for manipulating an image or operating an apparatus.

The display unit 160 and the display apparatus 1400 may be constituted by the same display. In other words, a single display may assume the functions of both the display unit 160 and the display apparatus 1400.

<Input Unit 170>

The input unit 170 is an operation console constituted by a mouse, a keyboard, or the like that can be operated by the user, for example. Alternatively, the display unit 160 may be constituted by a touch panel so that the display unit 160 can be used as the input unit 170.

The input unit 170 and the input apparatus 1500 may be constituted by the same apparatus. In other words, a single apparatus may assume the functions of both the input unit 170 and the input apparatus 1500.

<Introduction Unit 190>

The introduction unit 190 is configured to be capable of introducing a contrast agent into the interior of the subject 100 from the exterior of the subject 100. The introduction unit 190 may include a container storing the contrast agent and an injection needle that is inserted into the subject, for example. The introduction unit 190 is not limited thereto, however, and may take various forms as long as the contrast agent can be introduced thereby into the subject 100. In this case, the introduction unit 190 may be a well-known injection system, injector, or the like, for example. Further, the contrast agent may be introduced into the subject 100 by having the computer 150, acting as a control apparatus, control the operation of the introduction unit 190. Alternatively, the contrast agent may be introduced into the subject 100 by having the user operate the introduction unit 190.

<Subject 100>

The subject 100, although not a constituent element of the system, will now be described. The system according to this embodiment can be used for the purpose of diagnosing a malignant tumor, a vascular disease, or the like, observing the progress of chemotherapy, and so on in a human or an animal. Hence, the subject 100 is assumed to be a living body or organism, or more specifically a diagnosis target site such as a breast or one of various organs, the vascular network, the head, the neck, the trunk, or a limb, including the hands and feet, of a human or an animal. For example, when the measurement target is a human body, the target light absorber is oxyhemoglobin or deoxyhemoglobin, a blood vessel containing a large amount of oxyhemoglobin or deoxyhemoglobin, or a new blood vessel formed near a tumor, or the like. The target light absorber may also be plaque on the carotid wall or the like, or melanin, collagen, lipids, or the like contained in skin and so on. Further, a light absorber may be the contrast agent introduced into the subject 100. Contrast agents used in photoacoustic imaging include a pigment such as indocyanine green (ICG) or methylene blue (MB), fine gold particles, a mixture thereof, a substance acquired by collecting or chemically modifying these elements and introduced from the outside, and so on. The subject 100 may also be a phantom emulating a living organism.

The respective components of the photoacoustic apparatus may be formed as separate apparatuses or integrated into a single apparatus. Further, at least some of the configurations of the photoacoustic apparatus may be integrated into a single apparatus.

The respective apparatuses constituting the system according to this embodiment may be formed from separate hardware, or all of the apparatuses may be formed from a single piece of hardware. The functions of the system according to this embodiment may be realized by any type of hardware.

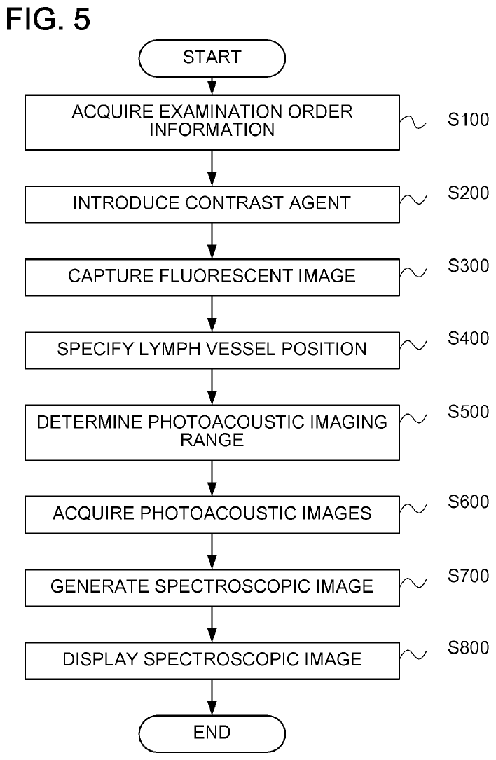

Next, using a flowchart shown in FIG. 5, an image generation method according to this embodiment will be described. The flowchart shown in FIG. 5 includes both steps performed by the system according to this embodiment and steps performed by a user such as a physician.

<S100: Step for Acquiring Examination Order Information>

The computer 150 of the photoacoustic apparatus 1100 acquires examination order information transmitted from an information system in a hospital, such as an HIS (Hospital Information System) or an RIS (Radiology Information System). The examination order information includes information such as the modality to be used in the examination, the contrast agent to be used in the examination, and so on.

<S200: Step for Introducing Contrast Agent>

The introduction unit 190 introduces the contrast agent into the subject. When the user introduces the contrast agent into the subject using the introduction unit 190, the user may operate the input unit 170 in order to transmit a signal indicating that the contrast agent has been introduced to the computer 150 functioning as a control apparatus from the input unit 170. Alternatively, the introduction unit 190 may transmit a signal indicating that the contrast agent has been introduced into the subject 100 to the computer 150. Further, the computer 150 stores the position on the subject 100 in which the contrast agent was introduced. The contrast agent may be administered to the subject without using the introduction unit 190. For example, the contrast agent may be administered by having the organism serving as the subject aspirate a sprayed contrast agent.

The subsequent step S300, to be described below, may be executed after waiting for a certain time to allow the contrast agent to reach the contrast enhancement target inside the subject 100 following introduction of the contrast agent.

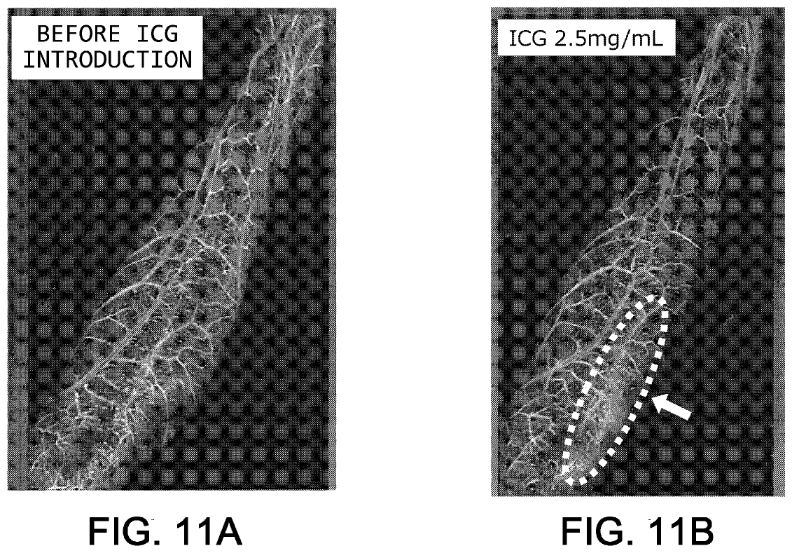

Here, a spectroscopic image acquired by imaging an organism into which ICG has been introduced using a photoacoustic apparatus will be described. FIGS. 11A and 11B to FIGS. 13A and 13B show spectroscopic images acquired by image capture when ICG is introduced in varying concentrations. In all of the image capture operations, 0.1 mL of ICG per location was introduced either subcutaneously or intradermally into the hand or foot. The subcutaneously or intradermally introduced ICG is taken into the lymph vessels only, and therefore the lumina of the lymph vessels are subjected to contrast enhancement. Further, in all of the image capture operations, the images were captured within 5 to 60 minutes following introduction of the ICG. Furthermore, all of the spectroscopic images were generated from photoacoustic images acquired by irradiating the organism with light having a wavelength of 797 nm and light having a wavelength of 835 nm.

FIG. 11A shows a spectroscopic image of a right forearm extensor when no ICG was introduced. FIG. 11B, meanwhile, shows a spectroscopic image of the right forearm extensor when ICG was introduced at a concentration of 2.5 mg/mL. Lymph vessels are depicted in a region indicated by a dotted line and an arrow in FIG. 11B.

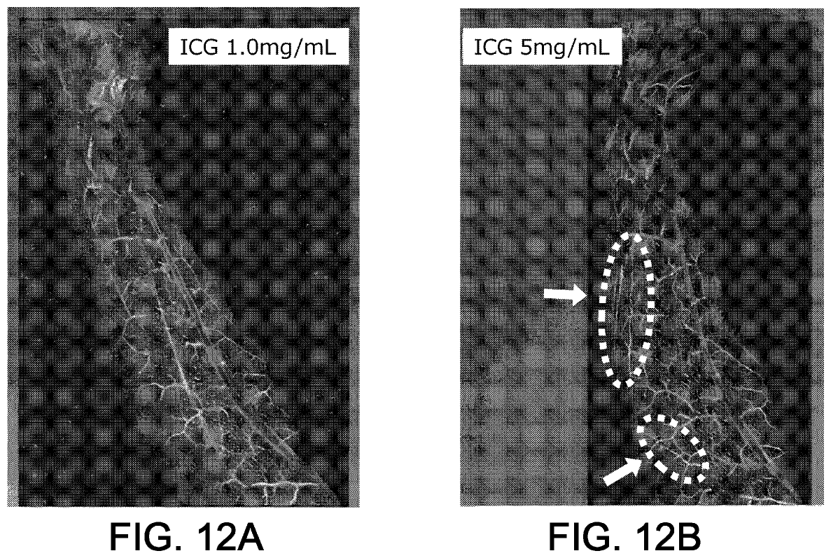

FIG. 12A shows a spectroscopic image of a left forearm extensor when ICG was introduced at a concentration of 1.0 mg/mL. FIG. 12B shows a spectroscopic image of the left forearm extensor when ICG was introduced at a concentration of 5.0 mg/mL. Lymph vessels are depicted in regions indicated by dotted lines and arrows in FIG. 12B.

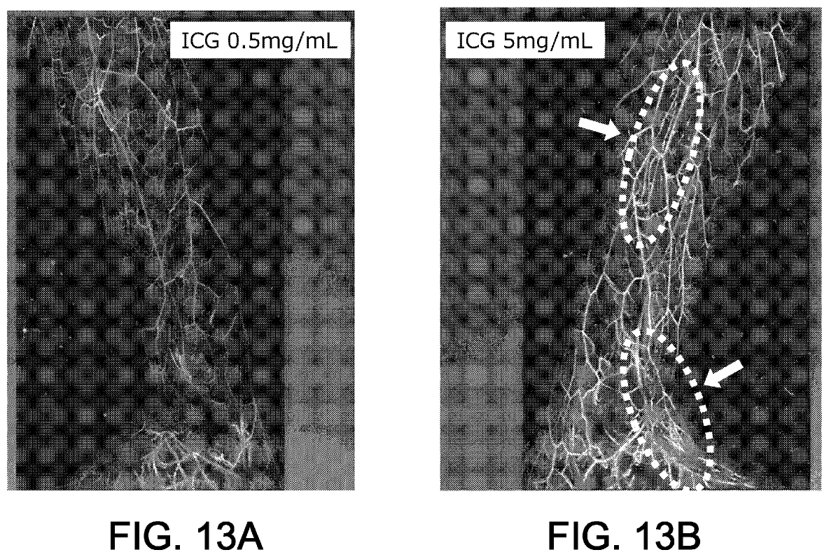

FIG. 13A shows a spectroscopic image of the inside of a lower right thigh when ICG was introduced at a concentration of 0.5 mg/mL. FIG. 13B shows a spectroscopic image of the inside of a lower left thigh when ICG was introduced at a concentration of 5.0 mg/mL. Lymph vessels are depicted in a region indicated by dotted lines and arrows in FIG. 13B.

It is evident from the spectroscopic images shown in FIGS. 11A and 11B to FIGS. 13A and 13B that when the concentration of the ICG increases, the visibility of the lymph vessels on the spectroscopic image improves. It is also evident from FIGS. 11A and 11B to FIGS. 13A and 13B that lymph vessels can be depicted in good quality when the concentration of the ICG equals or exceeds 2.5 mg/mL. In other words, striated lymph vessels can be seen clearly when the concentration of the ICG equals or exceeds 2.5 mg/mL. Therefore, when ICG is used as the contrast agent, the concentration thereof may be set to equal or exceed 2.5 mg/mL. In consideration of dilution of the ICG inside the organism, the concentration of the ICG may be set to exceed 5.0 mg/mL. Due to the solubility of Diagnogreen, however, it is difficult to dissolve ICG in an aqueous solution at a concentration equaling or exceeding 10.0 mg/mL.

Hence, the concentration of the ICG introduced into the organism is preferably between 2.5 mg/mL and 10.0 mg/mL, inclusive, and more preferably between 5.0 mg/mL and 10.0 mg/mL, inclusive.

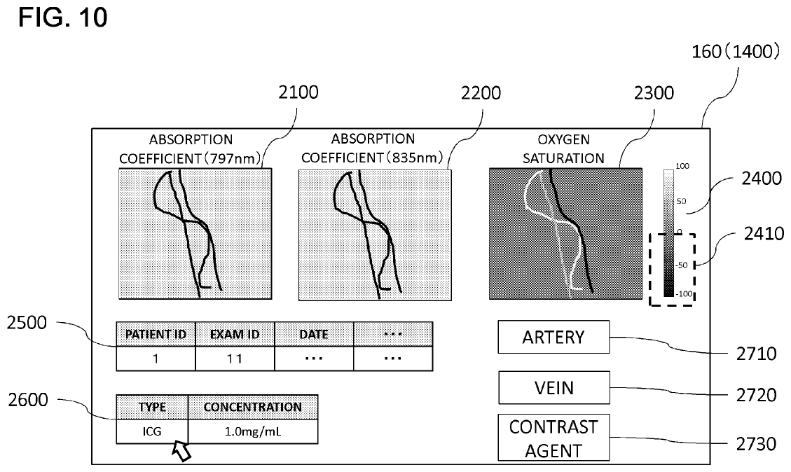

Accordingly, when ICG is designated as the contrast agent on an item 2600 on a GUI shown in FIG. 10, the computer 150 may only receive an instruction from the user indicating an ICG concentration within the numerical value range described above. In other words, in this case, it is not necessary for the computer 150 to receive instructions from the user indicating an ICG concentration outside the numerical value range described above. Hence, having acquired information indicating ICG as the contrast agent, the computer 150 no longer needs to receive instructions from the user indicating an ICG concentration lower than 2.5 mg/mL or higher than 10.0 mg/mL. Alternatively, having acquired information indicating ICG as the contrast agent, the computer 150 no longer needs to receive instructions from the user indicating an ICG concentration lower than 5.0 mg/mL or higher than 10.0 mg/mL.

The computer 150 may configure the GUI so that the user cannot indicate ICG concentrations outside the numerical value range described above on the GUI. In other words, the computer 150 may display the GUI so that the user cannot specify ICG concentrations outside the numerical value range described above on the GUI. For example, the computer 150 may display a pulldown menu on which ICG concentrations within the numerical value range described above can be specified selectively on the GUI. The computer 150 may configure the GUI so that on the pulldown menu, ICG concentrations outside the numerical value range described above are grayed-out and the grayed-out concentrations cannot be selected.

Further, when the user specifies an ICG concentration outside the numerical value range described above on the GUI, the computer 150 may issue an alert. Any method, such as displaying an alert on the display unit 160, issuing a sound, or illuminating a lamp, may be used as the alerting method.

Furthermore, when ICG is selected as the contrast agent on the GUI, the computer 150 may display the numerical value range described above on the display unit 160 as the concentration of the ICG to be introduced into the subject.

Note that the concentration of the contrast agent introduced into the subject is not limited to the numerical value range indicated here, and a suitable concentration for the purpose may be adopted. Further, an example in which the contrast agent is ICG was described here, but similar configurations to those described above may be used for other contrast agents.

By configuring the GUI in this manner, the user can be assisted in introducing the contrast agent into the subject at an appropriate concentration in accordance with the planned contrast agent to be introduced into the subject.

<S300: Step for Capturing Fluorescent Image>

The light emitting unit 115 irradiates the subject 100 with excitation light for exciting the contrast agent. Further, in conjunction with emission of the excitation light, the imaging unit 125 implements image capture, thereby acquiring a fluorescent image (a first image). Data of the fluorescent image captured by the imaging unit 125 are stored. During capture of the fluorescent image, the shutter 125a of the imaging unit 125 is opened, and when image capture is complete, the shutter 125a is closed.

When the target observation range is wider than the field of view of the imaging unit 125, fluorescent images of the entire target observation range are captured by performing image capture a plurality of times. At this time, fluorescent images of the entire target observation range may be captured before acquiring photoacoustic images, or fluorescent image capture and photoacoustic image acquisition may be performed in parallel. As will be described below, photoacoustic image acquisition is performed while varying the position of the probe 180 along the course of a lymph vessel, and therefore fluorescent image capture and photoacoustic image acquisition can be performed in parallel while moving the probe 180. The first fluorescent image should be captured so as to include the introduction position of the contrast agent.

<S400: Step for Specifying Lymph Vessel Position>

The computer 150 acquires position information indicating positions of lymph vessels and lymph nodes from the fluorescent image data. For example, the computer 150 specifies parts of the fluorescent image data having an image value (a luminance value) that exceeds a predetermined threshold as positions of lymph vessels and lymph nodes. From this position information, it is possible to identify the course pattern of a lymph vessel. In this embodiment, the computer 150 corresponds to a position information acquiring unit.

<S500: Determining Photoacoustic Image Capturing Range>

The computer 150 determines a photoacoustic image capturing range so as to include the position of a lymph vessel based on the position information acquired in S400. The order in which images are to be captured within the image capturing range may be determined as appropriate. For example, the computer 150 determines the image capturing range so that the contrast agent introduction position is set as a start position, image capture is performed along a course direction (the course) of a single lymph vessel, and when image capture of the single lymph vessel is complete, the computer 150 returns to the immediately preceding bifurcation position and performs image capture along the course direction of a different lymph vessel. The computer 150 determines the image capturing range and the image capturing order so that all lymph vessels are traced in this manner. Thus, the computer 150 can control the probe 180 on the basis of lymph vessel course information (information relating to the course of the lymph vessel, such as the position and course direction of the lymph vessel).

Note that when the fluorescent image does not include the entire target observation range, as described above, the computer 150 moves the probe 180 after capturing photoacoustic images, and then captures a fluorescent image in the new field of view and determines the image capturing range on the basis of the acquired fluorescent image.

In this embodiment, the computer 150 determines the image capturing range automatically, but the user may specify the image capturing range manually. More specifically, the computer may display the fluorescent image on the display unit 160 and receive an instruction from the user via the input unit 170 indicating the range of the fluorescent image in which to capture photoacoustic images.

In this embodiment, as described above, the computer 150 determines the measurement parameters (the image capturing range and so on) of photoacoustic measurement on the basis of the fluorescent image data.

<S600: Photoacoustic Image Acquisition>

The computer 150 controls the driving unit 130 to move the probe 180 (the light emitting unit 110 and the receiving unit 120) along the image capturing range determined in step S500. In other words, the driving unit 130 moves the probe 180 to a position in which a photoacoustic wave generated from the target position can be received. Hence, in this embodiment, the driving unit 130 controls the movement of the probe 180 so as to move the probe 180 along a lymph vessel determined from the fluorescent image data. In other words, in this embodiment, the photoacoustic measuring unit controls photoacoustic measurement on the basis of the fluorescent image data (the first image). Therefore, the aspect according to this embodiment may also be regarded as an aspect relating to a measurement control method for controlling photoacoustic measurement executed by a photoacoustic measuring unit.

A photoacoustic image (a second image) is acquired in each target position. As will be described in more detail below, photoacoustic image acquisition includes light emission from the light emitting unit 110, photoacoustic wave reception by the receiving unit 120, and photoacoustic image generation. Light of at least two wavelengths is emitted from the light emitting unit 110, and the light emission, photoacoustic wave reception, and photoacoustic image generation processes are executed at each wavelength.

When light is emitted from the light emitting unit 110, the computer 150 performs control so that the shutter 125a of the imaging unit 125 used to acquire the fluorescent image is closed.

A plurality of photoacoustic images may be generated in time series by acquiring photoacoustic images repeatedly a plurality of times in each position. In so doing, information relating to the flow of lymph can be acquired, and a moving image of photoacoustic images, and also spectroscopic images, can be displayed.

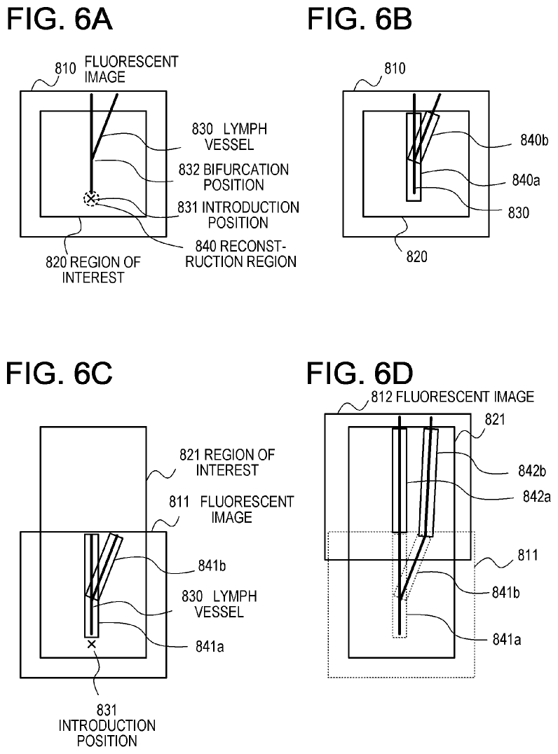

Photoacoustic image acquisition is performed over the entire image capturing range determined in step S500. FIG. 6A shows a fluorescent image 810 of a lymph vessel 830, a region of interest 820 that the user wishes to observe, and a reconstruction region 840, which is an imaging region of a photoacoustic image reconstructed by performing a single light emission operation. When the region of interest 820 is within the field of view of the single fluorescent image 810, as shown in FIG. 6A, image capturing ranges 840a and 840b in which to capture photoacoustic images are determined as shown in FIG. 6B on the basis of the position of the lymph vessel 830 appearing on the fluorescent image 810. The image capturing ranges 840a and 840b correspond to a region including a plurality of continuous reconstruction regions 840 formed by reconstructing photoacoustic images each time light is emitted during a plurality of light emission operations performed along the course of the lymph vessel 830.

First, photoacoustic images are captured in the image capturing range 840a that extends along the course of a single lymph vessel from a contrast agent introduction position 831. Once photoacoustic images have been captured up to the end of the region of interest 820, the probe 180 is returned to an immediately preceding bifurcation position 832, whereupon photoacoustic images are captured in the image capturing range 840b that extends along the course of the bifurcating lymph vessel. Thus, photoacoustic images are captured in all lymph vessel positions in the region of interest 820.

Photoacoustic measurement may be implemented by the photoacoustic measuring unit after adding a peripheral region extending along the course of the lymph vessel 830 appearing on the fluorescent image 810 to the image capturing range as well as the position of the lymph vessel 830. In so doing, it is possible to capture lymph vessels traveling through deep parts of the organism, for example, which are difficult to capture by fluorescence imaging but can be captured by photoacoustic imaging, and as a result, more detailed lymph vessel information is acquired. The photoacoustic apparatus may be switched to a mode in which the periphery of the lymph vessel is added to the image capturing range in response to an instruction from the user.

Next, a case in which a region of interest 821 does not fit into the field of view of a single fluorescent image, as shown in FIG. 6C, will be described. First, a fluorescent image 811 including the contrast agent introduction position 831 is captured. Then, image capturing ranges 841a and 841b in which to capture photoacoustic images are determined on the basis of the position of the lymph vessel 830 appearing on the fluorescent image 811. Photoacoustic images are first captured in the image capturing range 841a that extends along the course of a single lymph vessel from the contrast agent introduction position 831. When the image capturing position is moved, the image capturing range of the fluorescence imaging also changes. Therefore, when the reconstruction region 840 reaches the upper end of the fluorescent image 811, as shown in FIG. 6D, a second fluorescent image 812 can be captured. The fluorescent image 812 may be captured either at regular intervals or when it is necessary to extend the imaging positions in which photoacoustic images are to be captured. The course of the lymph vessel can be ascertained on the basis of the fluorescent image 812, and therefore an image capturing range 842a serving as an extension of the image capturing range 841a is determined. Once photoacoustic images have been captured up to the end of the region of interest 821 in the image capturing range 841a, the probe 180 is returned to the immediately preceding bifurcation position 832, whereupon photoacoustic images are captured in the image capturing range 841b that extends along the course of the bifurcating lymph vessel. Likewise with regard to the image capturing range 841b, an extended image capturing range 842b is determined on the basis of the second fluorescent image 812. Thus, photoacoustic images are captured in all lymph vessel positions in the region of interest 821.

<Wavelengths of Light Emitted During Photoacoustic Image Capture>

This embodiment generates a spectroscopic image having an image value corresponding to formula (1) in S700, as will be described below. From formula (1), an image value corresponding to the actual oxygen saturation in the blood vessel region of the spectroscopic image is calculated. However, the image value varies greatly according to the wavelength used in the contrast agent region of the spectroscopic image. Moreover, the image value varies greatly according to the absorption coefficient spectrum of the contrast agent in the contrast agent region of the spectroscopic image. As a result, the image value of the contrast agent region of the spectroscopic image may take a value that is indistinguishable from the image value of the blood vessel region. In order to ascertain the three-dimensional distribution of the contrast agent, however, the image value of the contrast agent region of the spectroscopic image preferably takes a value that can be distinguished from the image value of the blood vessel region.

Therefore, in this embodiment, the wavelengths of the emitted light used to capture photoacoustic images are preferably set at wavelengths at which the blood vessel region of the spectroscopic image can be distinguished from the contrast agent region. The wavelengths of the emitted light will be described below. Note that the wavelengths of the emitted light may be determined in advance in accordance with the contrast agent or determined dynamically by an information processing apparatus 300 on the basis of information relating to the contrast agent.

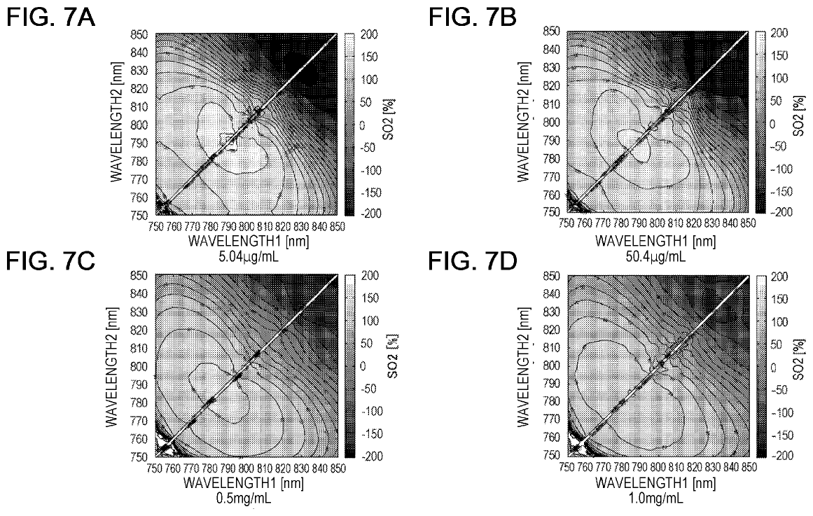

First described will be how the image value of the spectroscopic image corresponding to the contrast agent changes as a combination of wavelengths is modified. FIGS. 7A to 7D show results of a simulation of the image value (the oxygen saturation value) of the spectroscopic image corresponding to the contrast agent in respective combinations of two wavelengths. The vertical axes and the horizontal axes in FIGS. 7A to 7D represent a first wavelength and a second wavelength, respectively. FIGS. 7A to 7D show contours of the image value of the spectroscopic image corresponding to the contrast agent. FIGS. 7A to 7D respectively show the image value of the spectroscopic image corresponding to the contrast agent when the ICG concentration is set at 5.04 μg/mL, 50.04 μg/mL, 0.5 mg/mL, and 1.0 mg/mL. As shown in FIGS. 7A to 7D, depending on the combination of selected wavelengths, the image value of the spectroscopic image corresponding to the contrast agent may reach 60% to 100%. As noted above, when this combination of wavelengths is selected, it is difficult to distinguish the blood vessel region of the spectroscopic image from the contrast agent region. Therefore, a wavelength combination suitable for selection among those shown in FIGS. 7A to 7D, is a combination which makes the image value of the spectroscopic image corresponding to the contrast agent lower than 60% or higher than 100% shown in FIGS. 7A to 7D. Moreover, a wavelength combination suitable for selection among those shown in FIGS. 7A to 7D, is a combination which makes the image value of the spectroscopic image corresponding to the contrast agent be a negative value. For example, when ICG is used as the contrast agent, by selecting a wavelength of between 700 nm and 820 nm and a wavelength of between 820 nm and 1020 nm as the two wavelengths and generating a spectroscopic image from formula (1), the contrast agent region and the blood vessel region can be distinguished from each other clearly.

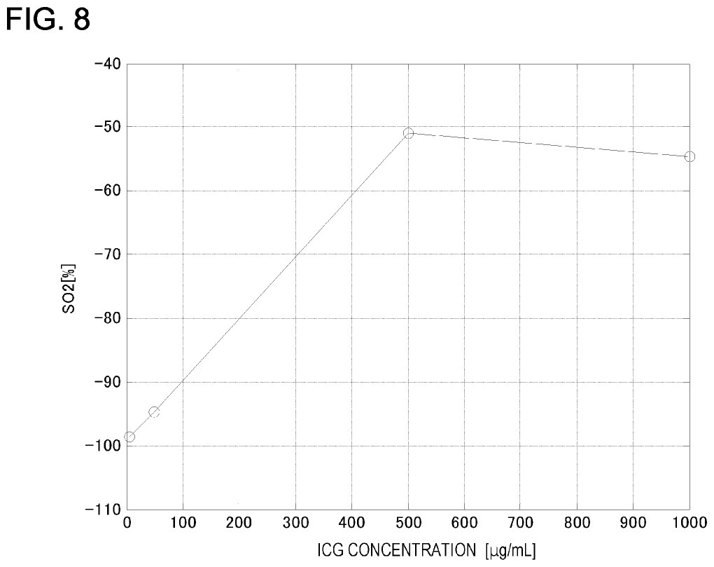

For example, here, considered will be a case in which 797 nm is selected as the first wavelength and 835 nm is selected as the second wavelength. FIG. 8 is a graph showing a relationship between the concentration of the ICG and the image value (the value of formula (1)) of the spectroscopic image corresponding to the contrast agent when 797 nm is selected as the first wavelength and 835 nm is selected as the second wavelength. According to FIG. 8, when 797 nm is selected as the first wavelength and 835 nm is selected as the second wavelength, the image value of the spectroscopic image corresponding to the contrast agent takes a negative value at any concentration between 5.04 μg/mL and 1.0 mg/mL. Hence, a spectroscopic image generated using this combination of wavelengths distinguishes the blood vessel region and the contrast agent region from each other clearly, since the oxygen saturation value of the blood vessels does not take a negative value in principle.

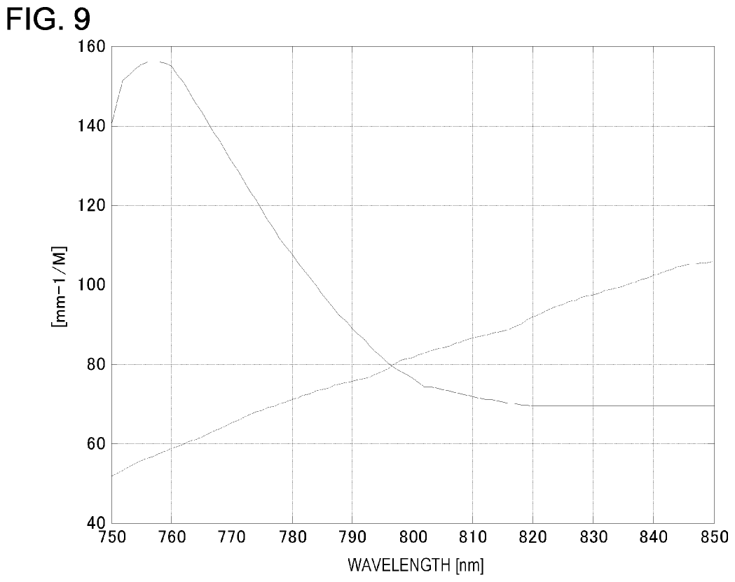

In the above description, the wavelengths are determined on the basis of information relating to the contrast agent, but the absorption coefficient of hemoglobin may also be taken into account for determining the wavelengths. FIG. 9 shows respective spectra of the molar absorption coefficient (dotted line) of oxyhemoglobin and the molar absorption coefficient (solid line) of deoxyhemoglobin. In a wavelength range shown in FIG. 9, the magnitude relationship between the molar absorption coefficient of oxyhemoglobin and the molar absorption coefficient of deoxyhemoglobin reverses about 797 nm. In other words, at shorter wavelengths than 797 nm, veins are easier to ascertain, and at longer wavelengths than 797 nm, arteries are easier to ascertain. Incidentally, lymphedema is treated by lymphovenous anastomosis, in which a bypass is created between a lymph vessel and a vein. In order to perform a preoperative examination, images of both a vein and a lymph vessel in which the contrast agent has accumulated may be formed by photoacoustic imaging. In this case, by making at least one of the plurality of wavelengths smaller than 797 nm, a clearer image of the vein can be formed. Moreover, making at least one of the plurality of wavelengths a wavelength at which the molar absorption coefficient of deoxyhemoglobin is larger than the molar absorption coefficient of oxyhemoglobin is advantageous in terms of forming an image of the vein. Furthermore, when a spectroscopic image is generated from photoacoustic images corresponding to two wavelengths, selecting wavelengths at which the molar absorption coefficient of deoxyhemoglobin is larger than the molar absorption coefficient of oxyhemoglobin as both wavelengths is advantageous in terms of forming an image of the vein. By selecting the wavelengths in this manner, images of both the vein and the lymph vessel into which the contrast agent has been introduced can be formed precisely during the preoperative examination performed prior to lymphovenous anastomosis.

When all of the plurality of wavelengths are wavelengths at which the absorption coefficient of the contrast agent is larger than that of blood, the oxygen saturation precision of the blood decreases due to an artifact derived from the contrast agent. Therefore, to reduce artifacts derived from the contrast agent, at least one of the plurality of wavelengths may be a wavelength at which the absorption coefficient of the contrast agent is smaller than the absorption coefficient of blood.

Here, a case in which a spectroscopic image is generated in accordance with formula (1) was described, but the present disclosure may also be applied to a case in which a spectroscopic image is generated so that the image value of the spectroscopic image corresponding to the contrast agent varies in accordance with conditions of the contrast agent and the wavelengths of the emitted light.

<Step for Emitting Light>

The step for emitting light within the photoacoustic image acquisition step S600 will now be described in more detail. The light emitting unit 110 sets the wavelengths determined in S400 in the light source 111. The light source 111 emits light at the wavelengths determined in S400. The light emitted from the light source 111 is emitted onto the subject 100 as pulsed light through the optical system 112. The pulsed light is absorbed in the interior of the subject 100, whereby a photoacoustic wave is generated by the photoacoustic effect. The introduced contrast agent also absorbs the pulsed light and generates a photoacoustic wave. In conjunction with transmission of the pulsed light, the light emitting unit 110 may also transmit a synchronization signal to the signal collecting unit 140. Moreover, the light emitting unit 110 performs light emission in a similar manner with respect to each of the plurality of wavelengths. The shutter 125a of the imaging unit 125 may be closed in synchronization with the timing at which light is emitted from the light emitting unit 110.

The user may specify control parameters such as the emission conditions of the light emitting unit 110 (the repetition frequency, wavelengths, and so on of the emitted light) and the position of the probe 180 using the input unit 170. The computer 150 may set control parameters determined on the basis of the specifications made by the user. The computer 150 may also move the probe 180 to the specified position by controlling the driving unit 130 on the basis of the specified control parameters. When image capture is specified in a plurality of positions, the driving unit 130 first moves the probe 180 to the first specified position. Note that when a measurement start instruction is issued, the driving unit 130 may move the probe 180 to a preprogrammed position.

<Step for Receiving Photoacoustic Wave>

The step for receiving a photoacoustic wave within the photoacoustic image acquisition step S600 will now be described in more detail. The signal collecting unit 140 begins a signal collection operation after receiving the synchronization signal transmitted from the light emitting unit 110. More specifically, the signal collecting unit 140 generates an amplified digital electric signal by amplifying and A/D-converting an analog electric signal derived from the photoacoustic wave and output by the receiving unit 120, and outputs the digital signal to the computer 150. The computer 150 stores the signal transmitted from the signal collecting unit 140. When multiple-time image capture is specified in a plurality of scanning positions, the light emission step and the photoacoustic wave reception step are executed repeatedly in the specified scanning positions so as to repeatedly emit pulsed light and generate digital signals derived from acoustic waves. The computer 150 may acquire and store information indicating the position of the receiving unit 120 at the time of light emission on the basis of the output of the position sensor of the driving unit 130 using light emission as a trigger.

In the above, a configuration in which a time-divided emission of the light of the plurality of wavelengths is exemplified, but as long as signal data corresponding respectively to the plurality of wavelengths can be acquired, the light emission method is not limited thereto. For example, light may be coded, and in this case the light of the plurality of wavelengths may be emitted substantially simultaneously.

<Step for Generating Photoacoustic Image>

The step for generating a photoacoustic image within the photoacoustic image acquisition step S600 will now be described in more detail. The computer 150 functioning as a photoacoustic image acquiring unit (the second image acquiring unit) generates a photoacoustic image on the basis of the stored signal data. The computer 150 outputs the generated photoacoustic image to the storage apparatus 1200 to be stored therein.

An analytical reconstruction method such as a time domain back projection method or a Fourier domain back projection method, or a model-based method (a repeated operation method), may be used as a reconstruction algorithm for converting the signal data into a two-dimensional or three-dimensional spatial distribution. Examples of time domain back projection methods include universal back-projection (UBP), filtered back-projection (FBP), phase addition (delay-and-sum), and so on.

In this embodiment, a single three-dimensional photoacoustic image (volume data) is generated by image reconstruction using a photoacoustic signal acquired by emitting light onto the subject once. Further, a time series of three-dimensional image data (a time series of volume data) is acquired by performing light emission a plurality of times and implementing image reconstruction each time light is emitted. Three-dimensional image data acquired by implementing image reconstruction for each of a plurality of light emission operations will be referred to collectively as three-dimensional image data corresponding to a plurality of light emission operations. Note that the plurality of light emission operations are executed in time series, and therefore the three-dimensional image data corresponding to the plurality of light emission operations constitute a time series of three-dimensional image data.

Furthermore, in this embodiment, the computer 150 generates a single set of three-dimensional image data by synthesizing the time series of three-dimensional image data. Moreover, in this embodiment, the relative positions of the subject and the receiving unit 120 are moved during light emission, and therefore the acquired synthesized three-dimensional image data encompass a wide range of the subject.

The computer 150 generates initial acoustic pressure distribution information (the acoustic pressures generated in a plurality of positions) as a photoacoustic image by executing reconstruction processing on the signal data. Further, the computer 150 may acquire absorption coefficient distribution information as a photoacoustic image by calculating a light fluence distribution of the light emitted onto the subject 100 in the interior of the subject 100 and dividing the initial acoustic pressure distribution by the light fluence distribution. A well-known method may be applied as a method for calculating the light fluence distribution. The computer 150 is also capable of generating photoacoustic images corresponding respectively to light of a plurality of wavelengths. More specifically, the computer 150 can generate a first photoacoustic image corresponding to a first wavelength by executing reconstruction processing on signal data acquired by emitting light at the first wavelength. Further, the computer 150 can generate a second photoacoustic image corresponding to a second wavelength by executing reconstruction processing on signal data acquired by emitting light at the second wavelength. In so doing, the computer 150 can generate a plurality of photoacoustic images corresponding to light of a plurality of wavelengths.

In this embodiment, the computer 150 acquires absorption coefficient distribution information corresponding respectively to light of a plurality of wavelengths as photoacoustic images. The absorption coefficient distribution information corresponding to the first wavelength is set as a first photoacoustic image, and the absorption coefficient distribution information corresponding to the second wavelength is set as a second photoacoustic image.

In the above, a configuration in which the system includes the photoacoustic apparatus 1100 for generating photoacoustic images is exemplified, but the present disclosure may also be applied to a system not including the photoacoustic apparatus 1100. As long as the image processing apparatus 1300 functioning as the photoacoustic image acquiring unit is capable of acquiring photoacoustic images, the present disclosure may be applied to any system. For example, the present disclosure may be applied to a system that includes the storage apparatus 1200 and the image processing apparatus 1300 but does not include the photoacoustic apparatus 1100. In this case, the image processing apparatus 1300 functioning as the photoacoustic image acquiring unit can acquire a photoacoustic image by reading a photoacoustic image specified from among photoacoustic images stored in advance in the storage apparatus 1200.

<S700: Step for Generating Spectroscopic Image>

The computer 150 functioning as a spectroscopic image acquiring unit generates a spectroscopic image on the basis of the plurality of photoacoustic images corresponding to a plurality of wavelengths. The computer 150 outputs the spectroscopic image to the storage apparatus 1200 to be stored in the storage apparatus 1200. As described above, the computer 150 may generate, as the spectroscopic image, an image indicating information corresponding to the concentration of a constituent substance of the subject, such as the glucose concentration, the collagen concentration, the melanin concentration, the volume fraction of fat or water, and so on. Further, the computer 150 may generate, as the spectroscopic image, an image representing a ratio of the first photoacoustic image corresponding to the first wavelength to the second photoacoustic image corresponding to the second wavelength. In this embodiment, an example in which the computer 150 generates an oxygen saturation image in accordance with formula (1) using the first photoacoustic image and the second photoacoustic image as the spectroscopic image is described.

Note that the image processing apparatus 1300 functioning as the spectroscopic image acquiring unit may acquire a spectroscopic image by reading a spectroscopic image specified from among spectroscopic images stored in advance in the storage apparatus 1200. Further, the image processing apparatus 1300 functioning as the photoacoustic image acquiring unit may acquire a photoacoustic image by reading at least one of the plurality of photoacoustic images used to generate the read spectroscopic image from a group of photoacoustic image stored in advance in the storage apparatus 1200.

<S800: Step for Displaying Image>

The image processing apparatus 1300 functioning as a display control unit displays the spectroscopic image on the display apparatus 1400 on the basis of the information relating to the contrast agent so that the region corresponding to the contrast agent can be distinguished from other regions. Image rendering method is not limited to any particular method, and any method, such as maximum intensity projection (MIP), volume rendering, or surface rendering, may be used. Here, rendering settings, such as the display region and the sight line direction used for rendering a three-dimensional image in two dimensions, may be specified as desired in accordance with the observation subject.

Here, described is a case in which 797 nm and 835 nm are set in S400 and a spectroscopic image is generated in accordance with formula (1) in S800. As shown in FIG. 8, when these two wavelengths are selected, the image value corresponding to the contrast agent on the spectroscopic image generated in accordance with formula (1) takes a negative value regardless of the ICG concentration.

As shown in FIG. 10, the image processing apparatus 1300 displays a color bar 2400 on the GUI as a color scale indicating a relationship between the image value of the spectroscopic image and the display color. The image processing apparatus 1300 may determine a numerical value range of image values to be allocated to the color scale on the basis of information relating to the contrast agent (information indicating that the contrast agent is ICG, for example) and information indicating the wavelengths of the emitted light. For example, the image processing apparatus 1300 may determine a numerical value range that includes the oxygen saturation of the vein, the oxygen saturation of the artery, and the negative image value corresponding to the contrast agent, acquired from formula (1). The image processing apparatus 1300 may determine a numerical value range of −100% to 100% and set the color bar 2400 by allocating −100% to 100% to a color gradation that varies from red to blue. With this display method, in addition to distinguishing veins from arteries, the contrast agent region having negative value can also be distinguished. The image processing apparatus 1300 may also display an indicator 2410 indicating a numerical value range of the image value corresponding to the contrast agent on the basis of the information relating to the contrast agent and the information indicating the wavelengths of the emitted light. Here, the negative-value region of the color bar 2400 is indicated by the indicator 2410 as the numerical value range of the image value corresponding to ICG. By displaying a color scale in such a manner that the display colors corresponding to the contrast agent can be distinguished, the region of the spectroscopic image corresponding to the contrast agent can be distinguished easily.

The image processing apparatus 1300 functioning as a region determining unit may determine the region of the spectroscopic image corresponding to the contrast agent on the basis of the information relating to the contrast agent and the information indicating the wavelengths of the emitted light. For example, the image processing apparatus 1300 may determine a region of the spectroscopic image having a negative image value as the region corresponding to the contrast agent. The image processing apparatus 1300 may then display the spectroscopic image on the display apparatus 1400 in such a manner that the region corresponding to the contrast agent can be distinguished from other regions. The image processing apparatus 1300 may identify or distinguish the region corresponding to the contrast agent by making the display color of the region corresponding to the contrast agent different to the display colors of the other regions, causing the region corresponding to the contrast agent to flash, or displaying an indicator (a frame, for example) indicating the region corresponding to the contrast agent.

Display mode may be switched, by selecting or pushing an item 2730 for ICG display on the GUI shown in FIG. 10, to a mode in which voxels or pixels having the image value corresponding to the ICG are selectively displayed. For example, when the user selects the item 2730 for ICG display, the image processing apparatus 1300 may display the ICG region selectively by selecting a voxel having a negative image value from the spectroscopic image and rendering the selected voxel selectively. Similarly, the user may select an item 2710 for artery display or an item 2720 for vein display. On the basis of the user's specification, the image processing apparatus 1300 may switch to a display mode for selectively displaying voxels or pixels having image values corresponding to arteries (between 90% and 100%, for example) or voxels or pixels having image values corresponding to veins (between 60% and 90%, for example). The numerical value ranges of the image values corresponding to arteries and the image values corresponding to veins can be modified on the basis of a user specification.

The displayed image may be generated by setting the image values of the spectroscopic image to at least one of hue, brightness, and chroma, and setting the image values of the photoacoustic image to the rest of hue, brightness, and chroma. For example, the generated image may have hue and chroma value according to the image value of the spectroscopic image, and have brightness value according to the image value of the photoacoustic image. When the displayed image is generated in this manner of setting the image value of the photoacoustic image to brightness, it may be difficult to visually identify both the blood vessels and the contrast agent, if the image value of the photoacoustic image corresponding to the contrast agent is by far larger or smaller than the image value of the photoacoustic image corresponding to the blood vessels. Therefore, it may be beneficial to modify a conversion table for converting the image value of the photoacoustic image to a brightness value in accordance with the image value of the spectroscopic image. For example, if the image value of the spectroscopic image falls within the numerical range corresponding to the contrast agent, the brightness corresponding to the image value of the spectroscopic image may be set larger compared with the case where the image value of the spectroscopic image falls within the numerical range corresponding to the blood vessels. In other words, if the image values of the photoacoustic image is same, the brightness value for the contrast agent region may be set larger than the brightness value for the blood vessel region. Furthermore, the numerical range of the photoacoustic image value which is not to be converted to brightness value may differ according to the image values of the spectroscopic image.

The conversion table may be modified as appropriate in accordance with the type and concentration of the contrast agent and the wavelengths of the emitted light. Accordingly, the image processing apparatus 1300 may determine a conversion table for converting the image values of the photoacoustic image into brightnesses on the basis of the information relating to the contrast agent and the information indicating the wavelengths of the emitted light. When the image value of the photoacoustic image corresponding to the contrast agent is estimated to be larger than the image value corresponding to the blood vessels, the image processing apparatus 1300 may make the brightness of the image value of the photoacoustic image corresponding to the contrast agent lower than the brightness of the image value of the photoacoustic image corresponding to the blood vessels. Conversely, when the image value of the photoacoustic image corresponding to the contrast agent is estimated to be smaller than the image value corresponding to the blood vessels, the image processing apparatus 1300 may make the brightness of the image value of the photoacoustic image corresponding to the contrast agent higher than the brightness of the image value of the photoacoustic image corresponding to the blood vessels.

The GUI shown in FIG. 10 displays an absorption coefficient image (the first photoacoustic image) 2100 corresponding to the 797 nm wavelength, an absorption coefficient image (the second photoacoustic image) 2200 corresponding to the 835 nm wavelength, and an oxygen saturation image (the spectroscopic image) 2300. The GUI may also display information indicating the wavelength of the light used to generate each image. In this embodiment, both a photoacoustic image and a spectroscopic image are displayed, but a spectroscopic image may be displayed alone. Further, the image processing apparatus 1300 may switch between photoacoustic image display and spectroscopic image display on the basis of a user specification.

<Synthesized Display of Fluorescent Image and Spectroscopic Image/Photoacoustic Image>

When displaying a spectroscopic image and/or a photoacoustic image, the image processing apparatus 1300 functioning as the display control unit may display the image so as to be synthesized with (superimposed on) a fluorescent image. Processing performed in a case where a photoacoustic image and a fluorescent image are synthesized and displayed will be described below. The image processing apparatus 1300 functioning as a synthesizing unit generates synthesized image data by synthesizing the fluorescent image data acquired in S300 with the photoacoustic image data acquired in S600. For example, the synthesized image is a superimposed image acquired by either superimposing the photoacoustic image on the fluorescent image or superimposing the fluorescent image on the photoacoustic image. The synthesized image may be an image acquired by arranging the fluorescent image and the photoacoustic image side by side.

The fluorescent image data and the photoacoustic image data may be synthesized using an identical weighting or filter in every imaging position (photoacoustic wave acquisition position), or may be synthesized by varying the weighting or filter in accordance with the imaging position. For example, the image processing apparatus 1300 may synthesize the fluorescent image data and the photoacoustic image data by modifying the weighting or the filter in accordance with the imaging position in a body axis direction. Further, the image processing apparatus 1300 may synthesize the fluorescent image data and the photoacoustic image data by varying the weighting or the filter in accordance with the distance between the contrast agent introduction position and the imaging position. The weighting or filter may be determined so that the contribution of the image value of the photoacoustic image increases as the distance between the imaging position and the contrast agent introduction position increases. This is to compensate the luminance reduction, or the decrease of luminance value of the photoacoustic image decreases according to the distance from the contrast agent introduction position. The distance between the imaging position and the contrast agent introduction position may be either a linear distance or a distance along the lymph vessel (the contrast enhancement target). Even when the photoacoustic image is displayed alone, the displayed luminance value of the photoacoustic image may be adjusted or corrected in accordance with the imaging position. Fluorescence imaging has higher sensitivity to the contrast agent than photoacoustic measurement, and therefore is able to observe all positions comparatively favorably. Hence, in order to suppress the processing amount of the image processing, it is preferable to modify the weighting or filter only for the photoacoustic image data in accordance with the position solely, without modifying the weighting or filter for the fluorescent image data in accordance with the position.

Furthermore, the fluorescent image and the photoacoustic image may be synthesized in such a manner that an imaging subject captured by fluorescence imaging can be distinguished from an imaging subject captured by photoacoustic imaging. For example, the image processing apparatus 1300 may display the region of the contrast enhancement subject captured on the fluorescent image in a different display color to the imaging subject captured by photoacoustic imaging (the contrast agent, i.e. lymph, lymph vessels, and veins/arteries). In so doing, a finding of dermal back flow (DBF), indicating that lymph is flowing back toward the skin, can be displayed so as to be distinguishable from interstitial leakage, which is rendered only by fluorescence imaging, and lymphangiectasis, which is rendered by both fluorescence imaging and photoacoustic imaging. Further, when a lymph vessel rendered in linear form by fluorescence imaging is not rendered by photoacoustic imaging, it is surmised that either the lymph flow is too fast or the contrast agent has been diluted, and the apparatus can notify the user of these possibilities. Moreover, regions rendered only on the fluorescent image and regions rendered on both the fluorescent image and the photoacoustic image may be displayed in different display colors. Fluorescence imaging captures lymph vessels, whereas photoacoustic imaging captures both lymph vessels and blood vessels, and therefore, lymph vessels and blood vessels can be displayed distinguishably on the photoacoustic image.

Synthesized display of a fluorescent image and a photoacoustic image was described here, but synthesized display of a fluorescent image and a spectroscopic image may be realized in a similar manner.

Furthermore, lymph flow information acquired from a time series of photoacoustic image may be displayed. Information relating to the flow speed (the movement speed) of the lymph may be cited as an example of the lymph flow information. The flow speed of the lymph can be determined by extracting the lymph vessel region from the spectroscopic image and acquiring the speed of luminance variation in the extracted region. The lymph vessel region can be extracted as a region of the spectroscopic image in which temporal variation in the luminance value is large. The speed of the luminance variation can be calculated on the basis of the frequency of luminance variation per unit time, for example the number of peaks (maximum values) of the luminance value within the unit time or the number of times the luminance value exceeds a predetermined threshold. The flow speed of the lymph may also be determined on the basis of the movement distance of the lymph per unit time.

Note that the display unit 160 may be made capable of displaying a moving image. For example, the image processing apparatus 1300 may generate at least one of the first photoacoustic image 2100, the second photoacoustic image 2200, and the spectroscopic image 2300 in time series, generate moving image data on the basis of the generated time series of images, and output the moving image data to the display unit 160. Note that in consideration of the fact that the flow frequency of lymph is comparatively low, the image data are preferably displayed as a static image or a time-compressed moving image in order to reduce the time necessary for the user's judgement. Moreover, by displaying a moving image, the lymph flow can be displayed repeatedly. The speed of the moving image may be set at a predetermined speed prescribed in advance or a predetermined speed specified by the user.

Furthermore, in the display unit 160 formed to be capable of displaying a moving image, the frame rate of the moving image is preferably variable. To make the frame rate variable, a window in which the user inputs the frame rate manually, a slide bar for modifying the frame rate, and so on may be added to the GUI shown in FIG. 10. Here, lymph flows through lymph vessels intermittently, and therefore only a part of the acquired time series of volume data can be used to check the lymph flow. Hence, when real-time display is performed to check the lymph flow, a reduction in efficiency may occur. Therefore, by making the frame rate of the moving image displayed on the display unit 160 variable, the displayed moving image can be displayed on fast-forward, and as a result, the user can check the fluid flow through the lymph vessels in less time.

The display unit 160 may also be made capable of repeatedly displaying a moving image within a predetermined time range. In such case, a GUI such as a window or a slide bar enabling the user to specify the range in which to perform repeated display is preferably added to FIG. 10. As a result, the user can easily ascertain the fluid flow through the lymph vessels, for example.

The method for displaying flow information is not limited to the methods described above. For example, the image processing apparatus 1300 functioning as the display control unit may display flow information relating to a specific region in association with the specific region on a single screen of the display apparatus 1400 using at least one method from among luminance display, color display, graph display, and numerical value display. Further, the image processing apparatus 1300 functioning as the display control unit may display at least one specific region in bold.

As described above, at least one of the image processing apparatus 1300 and the computer 150 functioning as an information processing apparatus may function as an apparatus including at least one of a spectroscopic image acquiring unit, a contrast agent information acquiring unit, a region determining unit, a photoacoustic image acquiring unit, and a display control unit. These units may respectively be constituted by different hardware or single pieces of hardware. Furthermore, a plurality of units may be constituted by a single piece of hardware.

In this embodiment, blood vessels can be distinguished from the contrast agent by selecting wavelengths at which the value of formula (1) corresponding to the contrast agent takes a negative value. The image value corresponding to the contrast agent, however, is not limited to a negative value but may take any value as long as the image value corresponding to the contrast agent enables blood vessels to be distinguished from the contrast agent. For example, the image processing described in this process can be applied to a case in which the image value of the spectroscopic image (the oxygen saturation image) corresponding to the contrast agent is smaller than 60% or larger than 100%, or the like.

In this embodiment, the photoacoustic apparatus 1100 includes a photoacoustic image capturing unit (a photoacoustic unit) and a fluorescent image capturing unit (a fluorescence imaging unit), but the subject information acquisition system may include a fluorescence imaging apparatus separately to the photoacoustic apparatus 1100. By operating the photoacoustic apparatus and the fluorescence imaging apparatus in conjunction, similar effects to those of the embodiment described above are acquired.

In this embodiment, a case in which ICG is used as the contrast agent was described as an example, but the image processing according to this embodiment may be applied to any contrast agent other than ICG. Moreover, the image processing apparatus 1300 may execute image processing corresponding to the contrast agent on the basis of information relating to the type of contrast agent to be introduced into the subject 100, from among a plurality of contrast agents.

In this embodiment, a case in which the image processing method is determined on the basis of acquired information relating to the used contrast agent, from among information relating to a plurality of contrast agents. Note, however, that when the conditions of the contrast agent to be used during image capture are uniquely determined, image processing corresponding to the conditions of the contrast agent may be set in advance. The image processing according to the embodiment described above can also be applied in this case.

In this embodiment, an example in which image processing is applied to a spectroscopic image based on photoacoustic images corresponding to a plurality of wavelengths was described, but the image processing according to this embodiment may also be applied to a photoacoustic image corresponding to a single wavelength. More specifically, the image processing apparatus 1300 may determine the region of the photoacoustic image corresponding to the contrast agent on the basis of the information relating to the contrast agent and display the photoacoustic image so that the region corresponding to the contrast agent can be distinguished from other regions. Further, the image processing apparatus 1300 may display a spectroscopic image or a photoacoustic image so that regions having a numerical value range of an image value corresponding to a preset contrast agent can be distinguished from other regions.

In this embodiment, an example in which the computer 150 functioning as the information processing apparatus generates a spectroscopic image by emitting light of a plurality of wavelengths was described, but in a case where a photoacoustic image is generated by emitting light of only one wavelength, the wavelength may be determined using the wavelength determination method according to this embodiment. In other words, the computer 150 may determine the wavelength of the emitted light on the basis of the information relating to the contrast agent. In this case, the computer 150 preferably determines the wavelength so that the image value of the contrast agent region of the photoacoustic image can be distinguished from the image value of the blood vessel region.

Note that the light emitting unit 110 may emit light of a preset wavelength onto the subject 100 so that the image value of the contrast agent region of the photoacoustic image can be distinguished from the image value of the blood vessel region. Further, the light emitting unit 110 may emit light of a plurality of preset wavelengths onto the subject 100 so that the image value of the contrast agent region of the spectroscopic image can be distinguished from the image value of the blood vessel region.

The image processing apparatus 1300 functioning as an image acquiring unit may acquire the fluorescent image data and the photoacoustic image data by reading the data from the group of images stored in the storage apparatus 1200 with reference to the supplementary information associated with the group of images. The image processing apparatus 1300 functioning as a fluorescent image acquiring unit (the first image acquiring unit) may acquire the fluorescent image data from the group of images stored in the storage apparatus 1200. Further, the image processing apparatus 1300 functioning as the photoacoustic image acquiring unit may acquire the photoacoustic image data from the group of images stored in the storage apparatus 1200. Furthermore, the image processing apparatus 1300 functioning as the synthesizing unit may generate synthesized image data by synthesizing fluorescent image data and photoacoustic image data read from the storage apparatus 1200.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-157800, filed on Aug. 24, 2018, which is hereby incorporated by reference herein in its entirety.

What is claimed is:

1. A system, comprising:
an image acquiring unit configured to acquire a first image generated by imaging fluorescence that is generated by emitting excitation light onto a subject into which a fluorescent contrast agent has been introduced; and
a photoacoustic measuring unit configured to implement photoacoustic measurement by receiving a photoacoustic wave generated in response to light emission onto the subject,
wherein the photoacoustic measuring unit is further configured to control the photoacoustic measurement on the basis of the first image.

2. The system according to claim 1, further comprising a position information acquiring unit configured to acquire position information indicating a position of a contrast enhancement target on the basis of the first image,
the photoacoustic measuring unit comprising:
a light emitting unit configured to emit light onto the subject;
a receiving unit configured to receive a photoacoustic wave; and
a moving unit configured to move the receiving unit on the basis of the position information to a position in which a photoacoustic wave generated from the contrast enhancement target can be received.

3. The system according to claim 2,
wherein the position information acquiring unit is further configured to acquire course information indicating a course direction of the contrast enhancement target on the basis of the first image, and
wherein the moving unit is further configured to move the receiving unit along the course direction of the contrast enhancement target.

4. The system according to claim 1, further comprising a fluorescence imaging unit configured to generate the first image by imaging fluorescence that is generated by emitting excitation light onto the subject into which a fluorescent contrast agent has been introduced.

5. The system according to claim 4, wherein the fluorescence imaging unit comprises:
an emitting unit configured to emit the excitation light;
an imaging unit configured to image the fluorescence; and
a light amount suppression unit configured to suppress an amount of light entering the imaging unit from the photoacoustic measuring unit in synchronization with a light emission timing.

6. An image processing apparatus, comprising:
a first image acquiring unit configured to acquire a first image generated by imaging fluorescence that is generated by emitting excitation light onto a subject into which a fluorescent contrast agent has been introduced;
a second image acquiring unit configured to acquire a second image generated on the basis of a photoacoustic wave that is generated in response to light emission onto the subject; and
a synthesizing unit configured to generate a synthesized image by synthesizing the first image with the second image.

7. The image processing apparatus according to claim 6, wherein the synthesizing unit is further configured to synthesize the first image with the second image by weighting the second image using a weighting corresponding to a position of the second image.

8. The image processing apparatus according to claim 6, further comprising a display control unit configured to display the synthesized image on a display unit.

9. The image processing apparatus according to claim 6, wherein the synthesized image is either a superimposed image of the first image and the second image or an image in which the first image and the second image are arranged side by side.

10. The image processing apparatus according to claim 6, wherein the second image is a photoacoustic image acquired by implementing reconstruction processing on a reception signal of the photoacoustic wave.

11. The image processing apparatus according to claim 6, wherein the second image is a spectroscopic image generated using photoacoustic signals that are based on photoacoustic waves generated by emitting light of a plurality of different wavelengths onto the subject and correspond respectively to the plurality of wavelengths.

12. A measurement control method, comprising:
acquiring a first image generated by imaging fluorescence that is generated by emitting excitation light onto a subject into which a fluorescent contrast agent has been introduced; and
controlling, on the basis of the first image, photoacoustic measurement in which a photoacoustic wave generated in response to light emission onto the subject is received.

13. The measurement control method according to claim 12, further comprising acquiring position information indicating a position of a contrast enhancement target on the basis of the first image,
wherein controlling the photoacoustic measurement comprising:
receiving a photoacoustic wave from the subject onto which light is emitted using a receiving unit; and
moving the receiving unit on the basis of the position information to a position in which a photoacoustic wave generated from the contrast enhancement target can be received.

14. The measurement control method according to claim 13, wherein acquiring the position information comprises acquiring course information indicating a course direction of the contrast enhancement target on the basis of the first image, and
in a step of moving the receiving unit, the receiving unit is moved along the course direction of the contrast enhancement target.

15. The measurement control method according to claim 12, further comprising generating the first image by imaging fluorescence that is generated by emitting excitation light onto the subject into which a fluorescent contrast agent has been introduced.

16. The measurement control method according to claim 15, wherein generating the first image comprises:
imaging the fluorescence from the subject onto which the excitation light is emitted; and
suppressing an amount of light entering an imaging unit configured to image the fluorescence in synchronization with a light emission timing.

17. An image processing method comprising:
acquiring a first image generated by imaging fluorescence that is generated by emitting excitation light onto a subject into which a fluorescent contrast agent has been introduced;
acquiring a second image generated on the basis of a photoacoustic wave that is generated in response to light emission onto the subject; and
generating a synthesized image by synthesizing the first image with the second image.

18. The image processing method according to claim 17, wherein, in a step of generating the synthesized image, the first image is synthesized with the second image by weighting the second image using a weighting corresponding to a position of the second image.

19. The image processing method according to claim 17, further comprising displaying the synthesized image on a display unit.

20. The image processing method according to claim 17, wherein the synthesized image is either a superimposed image of the first image and the second image or an image in which the first image and the second image are arranged side by side.

21. The image processing method according to claim 17, wherein the second image is a photoacoustic image acquired by implementing reconstruction processing on a reception signal of the photoacoustic wave.

22. The image processing method according to claim 17, wherein the second image is a spectroscopic image generated using photoacoustic signals that are based on photoacoustic waves generated by emitting light of a plurality of different wavelengths onto the subject and correspond respectively to the plurality of wavelengths.

23. A non-transitory computer-readable storage medium storing a program for causing a computer to execute the method according to claim 12.

24. A non-transitory computer-readable storage medium storing a program for causing a computer to execute the method according to claim 17.
